(19) United States
(12) Patent Application Publication
(10) Pub. No.: US 2022/0386936 A1
(43) Pub. Date: Dec. 8, 2022

(54) A SYSTEM, APPARATUS AND METHOD FOR MEASURING DYNAMIC VISUAL, VESTIBULAR AND SOMATOSENSORY ABILITY

(71) Applicant: UNIVERSITY OF CANBERRA, Australian Capital Territory (AU)

(72) Inventors: Gordon WADDINGTON, Bruce Canberra (AU); Braden MCGRATH, Bruce Canberra (AU)

(73) Assignee: UNIVERSITY OF CANBERRA, Australian Capital Territory (AU)

(21) Appl. No.: 17/774,905

(22) PCT Filed: Nov. 6, 2020
(86) PCT No.: PCT/AU2020/051205

§ 371 Date: May 6, 2022

(30) Foreign Application Priority Data
Nov. 6, 2019 (AU) 2019904174
Jun. 9, 2020 (AU) 2020901897

(51) Int. Cl. A61B 5/00; A61B 5/11; G16H 50/30; G16H 40/67

(52) U.S. Cl. CPC A61B 5/4023; A61B 5/1121; A61B 5/4595; A61B 5/6803; A61B 5/1114; A61B 5/702; A61B 5/7445; G16H 50/30; G16H 40/67; A61B 2562/0219; A61B 2560/0431

(57) ABSTRACT

An apparatus for making combined vestibular and somatosensory function assessments, comprising: a portable base unit comprising: a movable platform being at least partially rotatable about an axis of rotation; in use, a user stands with both feet on the movable platform; an adjustable stopping mechanism for adjusting an extent to which the movable platform can rotate in at least one direction with respect to a horizontal plane; and a controller for controlling the adjustable stopping mechanism to selectively adjust the extent to which the movable platform is rotated by one of a plurality of discrete measurable amounts based on a control signal; a visual occlusion headset worn by the user, the headset comprising a device for recording a vestibular function response in response to a vestibular function test.

### FIELD OF THE INVENTION

This present invention relates generally to a system, apparatus and method for measuring dynamic visual, vestibular and somatosensory ability. More particularly, but by no means exclusively, assessments made by the apparatus can be used to predict a user's athletic ability and the potential for future soft-tissue injury. Embodiments of the present invention can also be used as a rehabilitation tool for increasing vestibular and somatosensory acuity ability.

### BACKGROUND OF THE INVENTION

The somatosensory system is a part of the sensory nervous system, concerned with the conscious perception of touch, pressure, pain, temperature, position, movement, and vibration. Somatosensory ability, which includes both proprioception and tactile senses, underlies all human movement activity and can be a critical component of human motor performance.

Somatosensory ability has been shown to be: affected by injury and rehabilitation (it is worse after injury and changes with rehabilitation); indicative of falls risk in older persons (poorer somatosensory scores are associated with increased falls risk); and can be indicative of sporting performance talent (better scores in athletes are associated with better performance rankings).

The Ankle Movement Extent Discrimination Assessment (AMEDA) system was developed by one of the present inventors, Gordon Waddington, in 1995 for assessing somatosensory ability. The AMEDA system enables assessment of human somatosensory ability by assessment of the individual's ability to detect small differences in the extent of movements undertaken at the ankle without visual input. The AMEDA method has been shown to have better ecological validity and relatively better test validity and data validity than existing methods of somatosensory assessment.

While the original AMEDA does provide benefits over other existing assessment systems, it is unable to discriminate for visual and vestibular contributions of the user. It is also incapable of measuring exteroceptive inefficiency. The present inventors have found that incorporation of such factors would greatly increase the somatosensory ability assessment.

### SUMMARY OF THE INVENTION

In accordance with a first aspect there is provided an apparatus for assessing ankle movement extent discrimination of a user, comprising: a portable base unit comprising: a fixed platform; a movable platform disposed adjacent the fixed platform and being at least partially rotatable about an axis of rotation; in use, the user stands on the portable base unit in an even weight bearing stance with a first foot placed on the fixed platform and the other foot placed on the movable platform such that a long axis of the other foot is aligned with the axis of rotation of the movable platform; an adjustable stopping mechanism for adjusting an extent to which the movable platform can rotate in at least one direction with respect to a horizontal plane; and a controller for controlling the adjustable stopping mechanism to selectively adjust the extent to which the movable platform is rotated by one of a plurality of discrete predefined amounts based on a control signal; and a visual occlusion headset worn by the user, the visual occlusion headset comprising a sensing device for sensing a user response following an adjustment made by the adjustable stopping mechanism, the user response being utilised to assess ankle movement discrimination of the user.

In an embodiment the sensing device comprises one or more sensors for determining a predefined visual response effected by the user, the predetermined visual response representative of the user's recognition of the extent of the rotation effected by the adjustable stopping mechanism.

In an embodiment the headset is further configured with a display for presenting instructions to the user responsive to the adjustment made by the adjustable stopping mechanism.

In an embodiment the full visual occlusion headset further comprises a vestibular function response recording device for recording a vestibular function response of the user following an adjustment made by the adjustable stopping mechanism, or in response to a test instructed via the headset.

In an embodiment the vestibular function response recording device comprises an inclinometer and or accelerometer for recording the position of the wearers head relative to the gravitational horizontal.

In an embodiment the portable base unit further comprises an inclinometer for measuring an angle of incline of the movable platform.

In accordance with a second aspect there is provided an apparatus for making combined vestibular and somatosensory function assessments, comprising: a portable base unit comprising: a fixed platform; a movable platform being at least partially rotatable about an axis of rotation; in use, a user stands on the portable base unit in an even weight bearing stance with a first foot placed on the fixed platform and the other foot placed on the movable platform such that a long axis of the other foot is aligned with the axis of rotation for the movable platform; an adjustable stopping mechanism for adjusting an angle of rotation for the movable platform with respect to a horizontal plane; and a controller for controlling the adjustable stopping mechanism to selectively adjust the extent to which the movable platform is rotated by one of a plurality of discrete measurable amounts; a visual occlusion headset worn by the user, the headset comprising a device for recording a vestibular function response of the user associated with a vestibular function test.

In an embodiment the vestibular function response recording device comprises an inclinometer and/or an accelerometer.

In an embodiment the vestibular function test comprises presenting the user with a stimulus either through the headset or movable platform and wherein outputs of the inclinometer and/or accelerometer are utilised to determine a deviation of the user's head from true vertical.

In an embodiment the full occlusion headset comprises a display for displaying instructions and/or feedback associated with the vestibular function test.

In an embodiment the vestibular function test is associated with an adjustment made by the adjustable stopping mechanism.

In accordance with a further aspect there is provided an apparatus for making combined vestibular and somatosensory function assessments, comprising: a portable base unit comprising: a movable platform being at least partially rotatable about an axis of rotation; in use, a user stands with both feet on the movable platform; an adjustable stopping mechanism for adjusting an extent to which the movable platform can rotate in at least one direction with respect to a horizontal plane; and a controller for controlling the adjustable stopping mechanism to selectively adjust the extent to which the movable platform is rotated by one of a plurality of discrete measurable amounts based on a control signal; a visual occlusion headset worn by the user, the headset comprising a device for facilitating a vestibular function test.

In an embodiment the device comprises an inclinometer and/or an accelerometer.

In an embodiment the full occlusion headset comprises a display for displaying instructions and/or feedback associated with the vestibular function test.

In an embodiment the vestibular function test is associated with an adjustment made by the adjustable stopping mechanism.

In accordance with yet another aspect there is provided an apparatus for assessing ankle movement extent discrimination of a user, comprising: a portable base unit comprising: a fixed platform; a movable platform disposed adjacent the fixed platform and being at least partially rotatable about an axis of rotation; in use, the user stands on the portable base unit in an even weight bearing stance with a first foot placed on the fixed platform and the other foot placed on the movable platform such that a long axis of the other foot is aligned with the axis of rotation for the movable platform; an adjustable stopping mechanism for adjusting an extent to which the movable platform can rotate in a single direction with respect to a horizontal plane; and a controller for controlling the adjustable stopping mechanism to selectively adjust the extent to which the movable platform is rotated by one of a plurality of discrete measurable amounts based on a control signal for making a somatosensory assessment.

In one embodiment the apparatus further comprises a handheld device for receiving a user input and/or response associated with the vestibular function test.

In accordance with a still further aspect there is provided an apparatus for making a vestibular assessment, comprising: a portable base unit comprising: a movable platform being at least partially rotatable about an axis of rotation, in use, a user stands with both feet on the movable platform; an inclinometer coupled to the movable platform and being able to record data representative of an angle of rotation for the movable platform; a controller for receiving data output by the inclinometer and for recording a time that the user is able to maintain their balance on the movable platform without exceeding a predefined angle of rotation during an assessment period, the data received and recorded by the controller utilised for generating a vestibular response score; a visual occlusion headset worn by the user.

In an embodiment the headset comprises a display device for displaying data to the user during the assessment period for use in generating a visual response score.

In an embodiment, in one stage of the vestibular assessment, the user stands with both feet shoulder width apart and positioned directly over the rotational axis of the movable platform. In another stage of the vestibular assessment, the user stands on the movable platform with both feet shoulder width apart and astride the axis of rotation for the movable platform.

In an embodiment the apparatus further comprises a handheld device for receiving a user input and/or response related to a vestibular function assessment utilising the visual occlusion headset and wherein the user input and/or response is additionally utilised in generating the vestibular response score.

In accordance with a further aspect there is provided an apparatus for assessing ankle movement extent discrimination of a user, comprising: a base unit comprising: a fixed platform; a movable platform disposed adjacent the fixed platform and being at least partially rotatable about an axis of rotation; in use, the user stands on the portable base unit in an even weight bearing stance with a first foot placed on the fixed platform and the other foot placed on the movable platform such that a long axis of the other foot is aligned with the axis of rotation for the movable platform; an adjustable stopping mechanism for adjusting an extent to which the movable platform can rotate with respect to a horizontal plane; and a controller for controlling the adjustable stopping mechanism to selectively adjust the extent to which the movable platform is rotated; and a visual device worn by the user.

In an embodiment the apparatus further comprises a device for recording a user response following an adjustment made by the adjustable stopping mechanism, the user response comprising an indication of the adjustment sensed by the user following a rotational adjustment made to the movable platform and wherein the user response is utilised to assess ankle movement discrimination of the user. The device for recording a user response may comprise a handheld device operated by the user. The device may alternatively be incorporated into the visual occlusion device worn by the user and configured such that it senses the user response based on an eye movement made by the user.

### BRIEF DESCRIPTION OF THE ACCOMPANYING DRAWINGS

Embodiments of the present invention will now be described, by way of example only, with reference to the accompanying drawings, in which:

FIG. 1 is a schematic of a system comprising a base unit, headset, and remote-control unit for implementing an embodiment of the present invention;

FIG. 2 is an exploded view of the portable base unit shown in FIG. 1;

FIG. 3 shows a user operating the apparatus of FIG. 1;

FIG. 4 is an example user interface implemented by the headset of FIG. 1;

FIGS. 5 & 6 are views illustrating various stances for performing a visual and vestibular assessment using the apparatus of FIG. 4; and

FIG. 7 is a schematic illustrating how visual, vestibular and somatosensory data may be combined to determine a composite performance score, in accordance with an embodiment.

### DETAILED DESCRIPTION OF EXEMPLARY EMBODIMENTS OF THE INVENTION

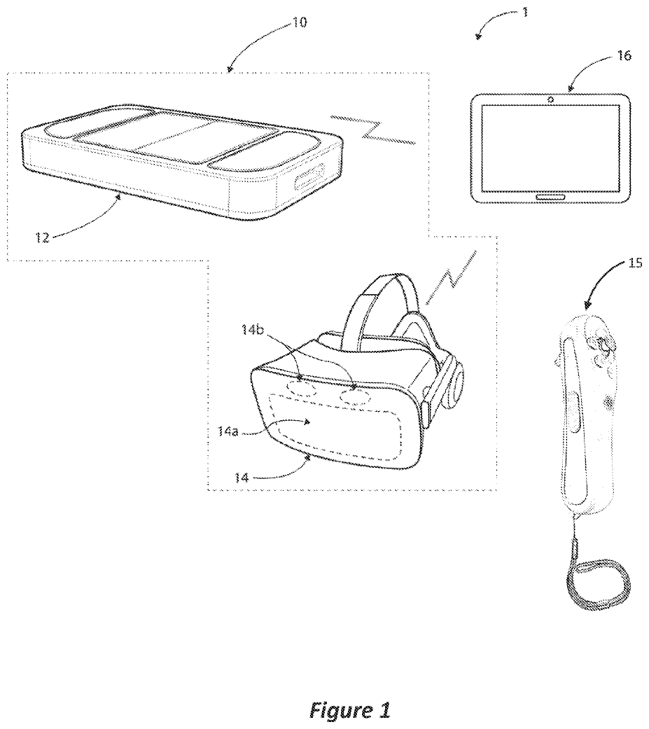

With reference to FIG. 1 there is shown a schematic illustration of a system 1 implementing an apparatus 10, in accordance with an embodiment of the invention. As will be described in detail in subsequent paragraphs, the apparatus 10 enables assessment of various visual, vestibular and somatosensory parameters. Assessments made by the apparatus 10 can advantageously be used for measuring a somatosensory contribution to human motor performance ability, that in turn can be evaluated to provide an indication of a user's athletic ability, as well as their potential for injury.

According to the illustrated embodiment, the apparatus 10 comprises a base unit 12 for performing the somatosensory assessment. A headset 14 is wearable by the user for registering user responses associated with the somatosensory assessment, as well as for performing vestibular function assessments relevant to the somatosensory assessment (as will be described in more detail in subsequent paragraphs). A remote control unit 16 is provided for receiving sensor output data from the base unit 12 and headset 14, as well as for generating control signals (e.g. for controlling moveable platform stopping position within the base unit 12, a display of the headset 14, etc). The remote control unit 16 may additionally be communicable with a hand-held remote 15 that allows the user to register responses to various assessments (i.e. in addition, or as an alternative to, aural responses or responses registered via the headset 14). The hand-held remote 15 may include any suitable input means for registering a user response. According to the illustrated embodiment, the remote includes depressible buttons assigned to predefined responses. According to the illustrated embodiment, the sensor data, user responses and control signals are wirelessly communicated by way of a Bluetooth connection, although it will be understood that these communications can be made using any suitable wired or wireless connection/communication protocol.

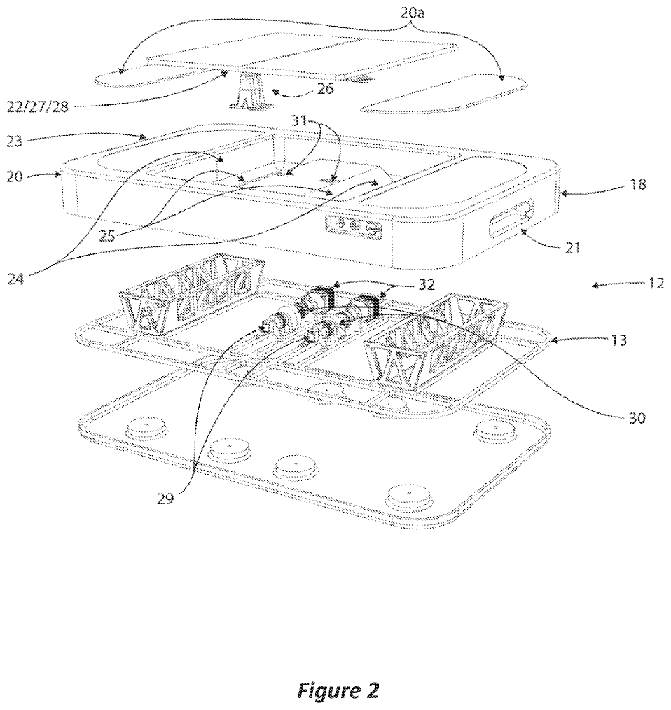

In more detail, and with additional reference to the exploded view of FIG. 2, the base unit 12 comprises an elongate outer housing 18. According to the illustrated embodiment, the base unit 12 is preferably sized and shaped so as to be readily portable. Fixed platform portions 20 are located adjacent first and second ends 21, 23 of the housing 18. To facilitate grip, a non-slip pad 20a is provided in a correspondingly shaped recess defined in an upper surface of the platform portion 20 at each end 21, 23. A movable platform 22 (also referred to as a “wobble board assembly”) is located adjacent in the centre of the unit 12 and has a rubberised textured upper surface that sits flush with the upper surface of the platform portion 20.

According to the illustrated embodiment, the movable platform 22 has a rectangular profile of aluminium construction, but may be comprised of any material with suitable properties. As shown, a longitudinal axis of the movable platform 22 is aligned with a longitudinal axis of the outer housing 18. The movable platform 22 is configured to seat within a recessed upper portion 24 of the outer housing 18 which has the same rectangular profile as the movable platform 22. An upper surface of the recessed portion slopes downwardly away from its midpoint (i.e. toward each end of the recess), thus creating voids 25 under corresponding portions of the movable platform 22. A gimble 26 mounted within the recessed portion 24 is centrally disposed under the movable platform 22 allowing it to pivot into either void 25 (i.e. to rotate about the platform's lateral axis in either direction).

An inclinometer 27 and accelerometer 28 are attached to, or disposed within, the movable platform 22. Readings from the inclinometer 27 and accelerometer 28 are wirelessly communicated to the remote control unit 16 by way of an electrically coupled Bluetooth module 29. It will be understood that the inclinometer output can be used to determine a relative position of the board, while the accelerometer output can be used to determine a change in velocity from one position to another.

An adjustable stopping mechanism is located in a subframe 13 of the base unit 12 for adjusting an extent to which the movable platform 22 can rotate into either void 25. Thus, a specific angle of rotation in either direction can be readily set for the platform as required for the assessment. In this instance the stopping mechanism comprises a pair of cams 30 that are mounted to the subframe 13 and which are each rotatable into and out of a corresponding void 25 through an opening 31. Each cam 30 is coupled to a servo motor assembly 32 for effecting its rotation. The cams 30 are rotatable to allow various discrete stopping points to be set for the movable platform 22, thus providing a suite of predefined ankle movement ranges when the movable platform is rotated from the horizontal (as will be described in more detail in subsequent paragraphs). The servo motor assemblies 32 are electrically connected to the Bluetooth module 29 for receiving wireless control signals from the remote control unit 16. In this regard, the remote control unit 16 can be controlled to issue a control signal for selectively adjusting the rotation of either cam 30 to achieve a desired stop position for the movable platform 22. As will be described in subsequent paragraphs, the desired stop position can be manually entered via the remote control unit 16, or automatically selected based on a control sequence.

Returning to FIG. 1, the headset 14 worn by the user takes the form of a visual occlusion headset comprising a visual display 14a and sensing device 14b for sensing a user response following a rotational adjustment of the movable platform 22. According to one embodiment, the sensing device comprises an eye tracker 14b having one or more sensors for determining a predefined visual response effected by the user (i.e. which is representative of the user's recognition of the movable board position). For example, the user might direct their gaze to a particular point on the display screen which registers a predefined movable board angle with respect to the horizontal (e.g. 1 degree, 2 degrees, 3 degrees, etc.). As another example, the user may to look to a particular point of the display screen 14a to indicate a lesser or greater angle of adjustment with respect to the previous adjustment. In a particular embodiment, the remote control unit 16 may be programmed with a computer program that iteratively creates a sequence of stop positions in response to the number of correct answers supplied by a user to enable dynamic movement sequences to be generated, thus optimising somatosensory testing functionality. In yet another alternative embodiment, the headset 14 may serve primarily to block out external stimulus, in which case the user may simply verbalise the adjustment/angle that they have sensed, which verbalised output is then recorded (either manually by the assessor or automatically by speech recognition software and hardware implemented by the headset or other suitable apparatus). As yet another alternative, the user may register their response via the hand-held remote 15. The headset 14 may additionally be configured with an inclinometer and/or accelerometer for recording a position of the user's head relative to the gravitational horizontal.

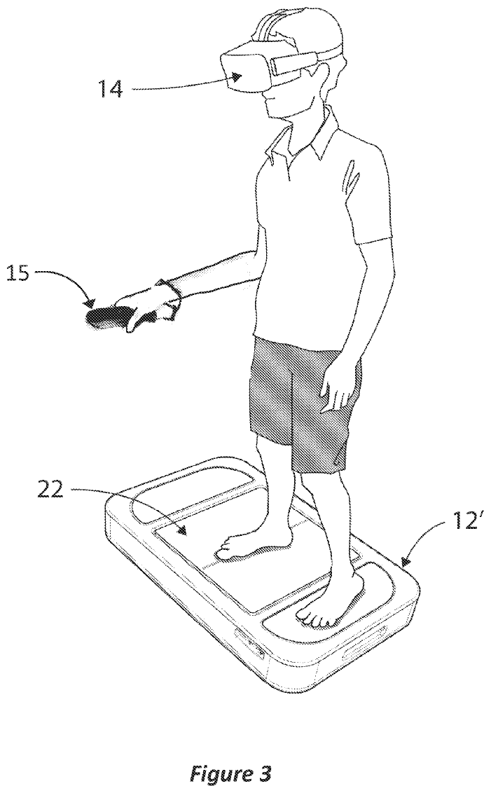

With additional reference to FIG. 3, a basic somatosensory assessment involves the user standing on the portable base unit 12 in an even weight bearing stance, with one foot placed on one of the fixed platforms 20 and the other foot placed on the midline of the movable platform 22 such that a long axis of each foot is aligned with the lateral axis of the movable platform 22. The cams 30 can be controlled so as to set a maximum angle of rotation for the movable platform 22 in either direction. It will be understood that the cams 30 may be controlled so that the movable platform 22 can only rotate in a single direction, depending on the particular assessment being carried out. The user recorded outputs (recorded either aurally, via the headset 14, or the hand-held remote 15) are then evaluated against the actual board angles for generating a somatosensory score. The somatosensory score may, for example, be a percentage score representative of how closely the user recorded outputs were to the actuals. Alternatively, the somatosensory score may be determined from a participant's discrimination between each pair of adjacent movement extents calculated using signal detection analysis generating areas under the response curves. It will be understood that various somatosensory ability scores may be derived and applied, depending on the desired implementation.

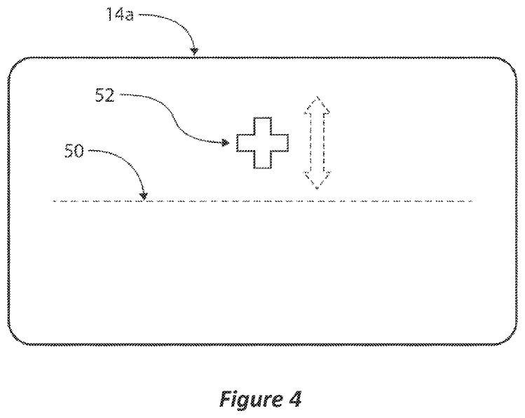

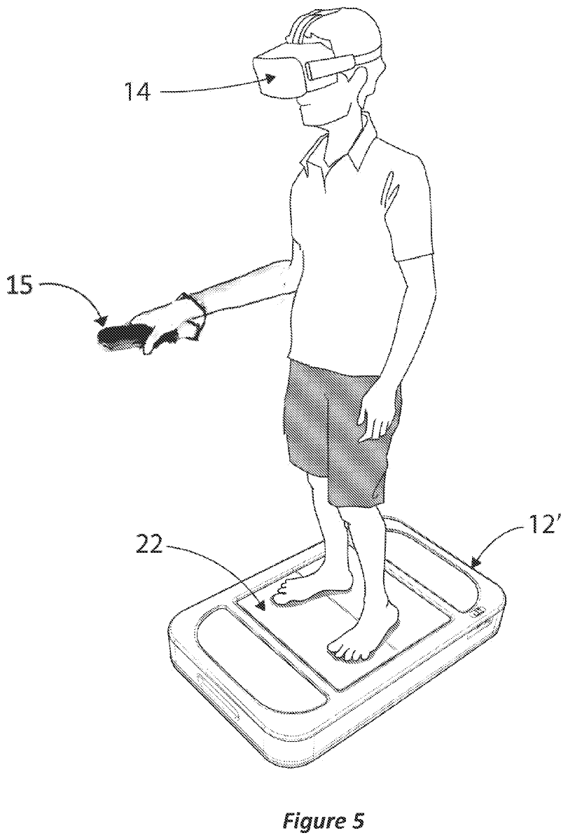

The contribution of visual function to upright stance can additionally be assessed by having the user maintain a cursor within a field of the headset display 14a, aligned as closely as possible to a fixed target line displayed in the centre of the headset display field. An example field of display is shown in FIG. 4, where the target line is denoted by reference numeral 50 and the curser denoted by reference numeral 52. The headset 14 is programmed such that the relative position of the cursor 52 within the headset display field is linked simultaneously to the position of the inclinometer 27 attached to the movable platform 22 of the portable base unit 12. The headset background display and target line 50 is referenced to the horizontal of the surface of the base unit 12.

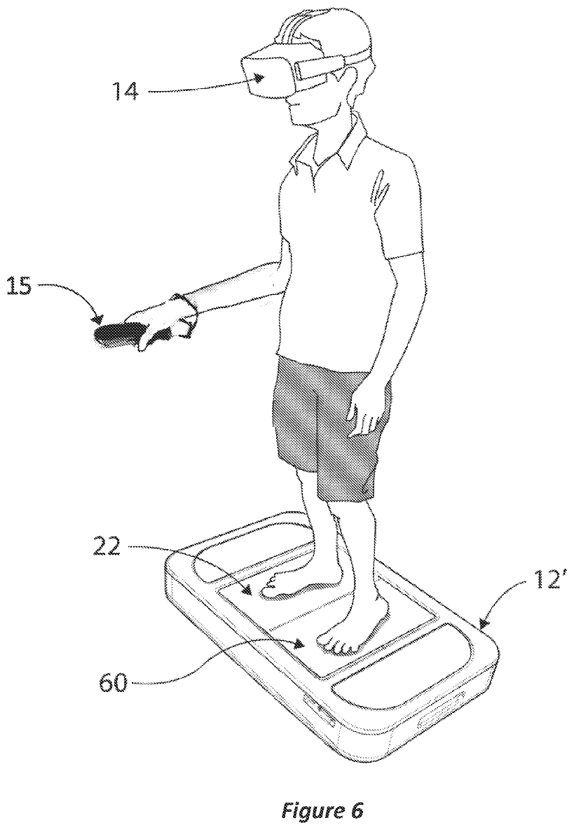

A form of combined vestibular, somatosensory assessment is performed in two stages. In a first stage, for anterior-posterior assessment, the user stands on the movable platform 22 with both feet shoulder width apart and positioned directly over the axis of rotation of the movable platform 22 (i.e. such the long axis of each foot is aligned perpendicular to the axis of rotation of the movable platform, in this instance the lateral axis). This is best shown in FIG. 5. In this position the user's body is undertaking a pitch movement. Once suitably positioned, the user is required to balance on the platform 22 to maintain its position as close to horizontal as possible, within predefined limits, for some predefined time, e.g. 20 seconds. The remote control unit 16 records a stability metric for the first assessment. In this example, the stability metric is the time that the user was able to maintain the movable platform 22 within 2.5 degrees of the horizontal (determined based on data output from the inclinometer 27). Data output from the accelerometer may also be recorded and evaluated.

In a second assessment stage, the user is instructed to stand on the movable platform 22 with both feet shoulder width apart and astride the axis of rotation such that their feet are aligned with the axis of rotation of the movable platform 22 (see FIG. 6). In this position the user's body is undertaking a roll movement. The remote control unit 16 then records the same stability metric as for the first stage (i.e. the time the user is able to maintain the movable platform within 2.5 degrees of horizontal). Again, acceleration data may be recorded for the second stage. The remote control unit 16 then determines an average stability measure for the two stages (using the data output from the inclinometer and accelerometer) and provides a feedback score against a normative data set. It will be understood that the feedback score may be based on only one of the stage assessment metrics, although this may result in a less meaningful output.

In a particular embodiment, the headset 14 may also be configured to measure a form of vestibular function. In this regard, the sensor device 14b comprises InfraRed (IR) eye tracking cameras within the headset which are calibrated to each user. Calibration software may, for example, involve the user looking at a green dot in four key directions; right, up, left and down. By way of example, one form of vestibular function may be assessed by making eye gaze evaluations, examples of which are described below. In this assessment the user is asked to keep their head still and to look straight ahead. The assessment begins with a dark background displayed on the headset display 14a. During the assessment, a moving target is displayed on the headset display 14a and the movement of the subject's pupils in response to a change in the location of the target is recorded by the IR cameras 14b and communicated to the remote control unit 16 for subsequent evaluation.

In a particular embodiment, the headset 14 may also be configured to measure an additional form of vestibular function. In this regard the sensor device comprises an inclinometer and/or accelerometer coupled to the headset 14 which measures the position of the user's head. The user is presented with a stimulus which could be visual through the headset display, or somatosensory through the platform, and asked to maintain their head in an upright position. The deviation of the head position from true vertical is measured and communicated to the remote control unit 16 for subsequent evaluation.

In an additional and/or alternative vestibular function assessment, the hand-held remote 15 may be utilised for recording a user input associated with a test presented via the headset 14. For example, the user may be asked to use the remote 15 to orient a line shown in the headset display so that it aligns with earth horizontal. For a combined vestibular somatosensory assessment, the user may be asked to use the hand-held remote 15 to orient a line shown in the headset display to be equal to the position of the moveable platform 22.

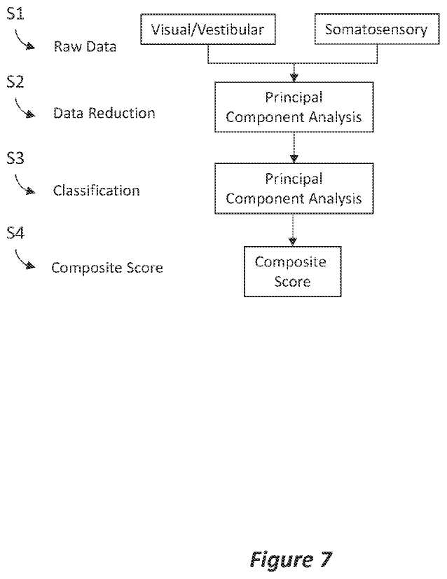

The visual, vestibular and somatosensory assessment data output using one or more of the above assessments may be combined to create a single Composite Performance Score (CPS), using a data reduction process as schematically illustrated in FIG. 7.

Combining and presenting this raw data in a single score may provide an easily interpretable measure avoiding the potential for information overload. It will be understood that the score may be calculated by the remote control unit, or some other suitable computer processing arrangement.

In more detail, at Step S1, the visual, vestibular and somatosensory data is gathered and input to a data reduction process, where the data gets condensed (step S2). By way of example, a Principal Component Analysis (PCA) may be used to reduce the raw input data. As persons skilled in the art will understand, PCA is a technique used to identify patterns in data, by mapping the raw data into a lower dimensional space. The resultant reduced data is provided as the input to a Support Vector Machine (SVM) machine learning classifier which, at step S4, calculates the CPS.

Using a headset 14 to record user response has several notable advantages. Firstly, the headset 14 occludes the user's vision, thereby preventing external stimulus that can adversely impact on the assessment. The headset 14 also allows the user to stand upright during the somatosensory assessment and discourages the user from looking at the portable base unit 12, which again can adversely impact on assessment. The headset 14 also provides an efficient and speedy interface for recording the user response.

It will be understood that the remote control unit 16 as described above could take the form of any suitable computing arrangement with the appropriate software and hardware for communicating with the portable base unit 12 and headset 14. For example, the remote control unit may take the form of a computer tablet, laptop computer or the like. In another form, the remote control unit 16 may be directly coupled or incorporated into any one of the portable unit 12, handheld remote 15 or headset 14. It will also be understood that the unit 12 may be configured with a fixed platform on only one side of the unit, without departing from the scope of the invention.

It will be understood that the axis of rotation for the movable platform 22 will typically be aligned with either the long or short axis of the base unit 12. However, it will be understood that this is not essential and that the axis of orientation may be at an angle with respect to the base unit 12.

The apparatus and methods described herein are not limited to the specific embodiments described herein but, rather, operations of the methods and/or components/modules of the various apparatus may be utilised independently and separately from other operations and/or components/modules described herein. Further, the described operations and/or components/modules may also be defined in, or used in combination with, other systems, methods, and/or apparatus, and are not limited to practice with only the systems, methods, and storage media as described herein.

Embodiments of the present disclosure may be described in the general context of computer-executable instructions, such as program components or modules, executed by one or more computers or other devices. Aspects of the present disclosure may be implemented with any number and organisation of components or modules. For example, aspects of the present disclosure are not limited to the specific computer executable instructions or the specific components or modules illustrated in the figures and described herein. Alternative embodiments of the present disclosure may include different computer-executable instructions or components having more or less functionality than illustrated and described herein.

A computer or controller, such as those described herein, includes at least one processor or processing unit and a system memory. The computer or controller typically has at least some form of computer readable media. By way of example and not limitation, computer readable media include computer storage media and communication media. Computer storage media include volatile and non-volatile, removable and non-removable media implemented in any method or technology for storage of information such as computer readable instructions, data structures, program modules, or other data. Communication media typically embody computer readable instructions, data structures, program modules, or other data in a modulated data signal such as a carrier wave or other transport mechanism and include any information delivery media. Those skilled in the art are familiar with the modulated data signal, which has one or more of its characteristics set or changed in such a manner as to encode information in the signal. Combinations of any of the above are also included within the scope of computer readable media.

In addition, although the invention has been described with reference to the present embodiments, it will be understood by those skilled in the art that alterations, changes and improvements may be made and equivalents may be substituted for the elements thereof and steps thereof without departing from the scope of the invention. Further, many modifications may be made to adapt the invention to a particular situation without departing from the central scope thereof. Such alterations, changes, modifications and improvements, though not expressly described above, are nevertheless intended and implied to be within the scope and spirit of the invention. The above described embodiments are therefore not to be taken as being limiting in any respects.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1-26: canceled

27. An apparatus for assessing ankle movement extent discrimination of a user, comprising:
a portable base unit comprising:
a fixed platform;
a movable platform disposed adjacent the fixed platform and being at least partially rotatable about an axis of rotation;
in use, the user stands on the portable base unit in an even weight bearing stance with a first foot placed on the fixed platform and the other foot placed on the movable platform such that a long axis of the other foot is aligned with the axis of rotation of the movable platform;
an adjustable stopping mechanism for adjusting an extent to which the movable platform can rotate in at least one direction with respect to a horizontal plane; and
a controller for controlling the adjustable stopping mechanism to selectively adjust the extent to which the movable platform is rotated by one of a plurality of discrete predefined amounts based on a control signal.

28. An apparatus in accordance with claim 27, further comprising a visual occlusion headset worn by the user.

29. An apparatus in accordance with claim 28, wherein the visual occlusion headset comprises a sensing device for sensing a user response following an adjustment made by the adjustable stopping mechanism, the user response being utilised to assess ankle movement discrimination of the user and wherein the sensing device comprises one or more sensors for determining a predefined visual response effected by the user, the predetermined visual response representative of the user's recognition of the extent of the rotation effected by the adjustable stopping mechanism.

30. An apparatus in accordance with claim 28, wherein the visual occlusion headset comprises a sensing device for sensing a user response following an adjustment made by the adjustable stopping mechanism, the user response being utilised to assess ankle movement discrimination of the user and wherein the headset is further configured with a display for presenting instructions to the user responsive to the adjustment made by the adjustable stopping mechanism.

31. An apparatus in accordance with claim 28, wherein the visual occlusion headset comprises a sensing device for sensing a user response following an adjustment made by the adjustable stopping mechanism, the user response being utilised to assess ankle movement discrimination of the user and wherein the full visual occlusion headset further comprises a vestibular function response recording device for recording a vestibular function response of the user following an adjustment made by the adjustable stopping mechanism, or in response to a test instructed via the headset.

32. An apparatus in accordance with claim 30, wherein the vestibular function response recording device comprises an inclinometer and or accelerometer for recording the position of the wearers head relative to the gravitational horizontal.

33. An apparatus in accordance with claim 27, wherein the portable base unit further comprises an inclinometer for measuring an angle of incline of the movable platform.

34. An apparatus in accordance with claim 28, further comprising a device for recording a user response following an adjustment made by the adjustable stopping mechanism, the user response comprising an indication of the adjustment sensed by the user following a rotational adjustment made to the movable platform and wherein the user response is utilised to assess ankle movement discrimination of the user and wherein the device is either a handheld device operated by the user or a device incorporated into the visual occlusion device and is configured to sense the user response based on an eye movement made by the user.

35. An apparatus for making combined vestibular and somatosensory function assessments, comprising:
a portable base unit comprising:
a fixed platform;
a movable platform being at least partially rotatable about an axis of rotation;
in use, a user stands on the portable base unit in an even weight bearing stance with a first foot placed on the fixed platform and the other foot placed on the movable platform such that a long axis of the other foot is aligned with the axis of rotation for the movable platform;
an adjustable stopping mechanism for adjusting an angle of rotation for the movable platform with respect to a horizontal plane; and
a controller for controlling the adjustable stopping mechanism to selectively adjust the extent to which the movable platform is rotated by one of a plurality of discrete measurable amounts;
a visual occlusion headset worn by the user, the headset comprising a device for recording a vestibular function response of the user associated with a vestibular function test.

36. An apparatus in accordance with claim 35, wherein the vestibular function response recording device comprises an inclinometer and/or an accelerometer and wherein the vestibular function test comprises presenting the user with a stimulus either through the headset or movable platform and wherein outputs of the inclinometer and/or accelerometer are utilised to determine a deviation of the user's head from true vertical.

37. An apparatus in accordance with claim 35, wherein the visual occlusion headset comprises a display for displaying instructions and/or feedback associated with the vestibular function test.

38. An apparatus in accordance with claim 37, wherein the vestibular function test is associated with an adjustment made by the adjustable stopping mechanism.

39. An apparatus for making combined vestibular and somatosensory function assessments, comprising:
a portable base unit comprising:
a movable platform being at least partially rotatable about an axis of rotation;
in use, a user stands with both feet on the movable platform;
an adjustable stopping mechanism for adjusting an extent to which the movable platform can rotate in at least one direction with respect to a horizontal plane; and
a controller for controlling the adjustable stopping mechanism to selectively adjust the extent to which the movable platform is rotated by one of a plurality of discrete measurable amounts based on a control signal;
a visual occlusion headset worn by the user, the headset comprising a device for facilitating a vestibular function test.

40. An apparatus in accordance with claim 39, wherein the device for facilitating the vestibular function test comprises an inclinometer and/or an accelerometer.

41. An apparatus in accordance with claim 39, wherein the visual occlusion headset comprises a display for displaying instructions and/or feedback associated with the vestibular function test and wherein the vestibular function test is associated with an adjustment made by the adjustable stopping mechanism.

42. An apparatus for making a vestibular assessment, comprising:
a portable base unit comprising:
a movable platform being at least partially rotatable about an axis of rotation, in use, a user stands with both feet on the movable platform;
an inclinometer coupled to the movable platform and being able to record data representative of an angle of rotation for the movable platform;
a controller for receiving data output by the inclinometer and for recording a time that the user is able to maintain their balance on the movable platform without exceeding a predefined angle of rotation during an assessment period, the data received and recorded by the controller utilised for generating a vestibular response score;
a visual occlusion headset worn by the user.

43. An apparatus in accordance with claim 42, wherein the headset comprises a display device for displaying data to the user during the assessment period for use in generating a visual response score.

44. An apparatus in accordance with claim 42, wherein, in one stage of the vestibular assessment, the user stands with both feet shoulder width apart and positioned directly over the rotational axis of the movable platform.

45. An apparatus in accordance with claim 44, wherein, in another stage of the vestibular assessment, the user stands on the movable platform with both feet shoulder width apart and astride the axis of rotation for the movable platform.

46. An apparatus in accordance with claim 42, further comprising a handheld device for receiving a user input and/or response related to a vestibular function assessment utilising the visual occlusion headset and wherein the user input and/or response is additionally utilised in generating the vestibular response score.
